(19) United States
(12) Patent Application Publication
(10) Pub. No.: US 2009/0131872 A1
(43) Pub. Date: May 21, 2009

(54) CATHETER PLACEMENT DEVICE

(76) Inventor: Sergey Popov, Beer Sheva (IL)

Correspondence Address: DANIEL J SWIRSKY, 55 REUVEN ST., BEIT SHEMESH, 99544, IL

(21) Appl. No.: 11/813,002

(22) PCT Filed: Dec. 26, 2005
(86) PCT No.: PCT/IL05/01377

§ 371 Date: Apr. 16, 2008

(30) Foreign Application Priority Data
Dec. 28, 2004 (IL) 166032

(51) Int. Cl. A61M 25/06

(52) U.S. Cl. 604/164.08; 604/164.01

(57) ABSTRACT

Abstract A catheter placement device (see FIG. 2) includes a needle unit, which in an initial position is releasably engaged with a housing distal end and has a sharp needle passing through a catheter and protruding distally of it. There is also a telescopic protector including guard unit, which is sliding in a housing guide. After inserting the needle into patient's vein, operator one-handedly pushes the guard unit distally, thereby emplacing the catheter, displacing the protector into a full length position and locking therein, and passively disengaging the needle unit from the hosing by a trigger cam located on the guard unit. As a result, a retracting spring moves a needle into retracted position, wherein it is protected from any contact with personal. Then, operator disconnects the guard from the catheter, turns the housing and occludes the catheter entrance with a cap, which was detachably mounted on the housing.

### BACKGROUND OF THE INVENTION

1. Field of the Invention

The present invention relates to intravenous catheter placement devices, which employ a sharp needle to introduce and emplace an intravenous catheter into a blood vessel of a patient, specifically, to the devices wherein the needle is retracted into a protected position after catheter emplacement to prevent the user from accidental pricking by contaminated needle.

2. Prior Art of the Invention

Nowadays the protection of the contaminated needle sharp point from contact with personnel after the catheter emplacement is one of the main demands to the intravenous catheter placement devices. In this connection, the many modern devices provide the needle sharp point protection carried out as an alienable part of the general process of the catheter placement. Specifically, U.S. Pat. No. 5,573,510 “Safety intravenous catheter assembly with automatically retractable needle” discloses one of such devices. It includes a safety receptacle containing a retractable needle and connected to the catheter through a coupling appliance, which is removably fastened to the catheter and has an elongated stem extending into the receptacle past a needle-stopping member within the receptacle. In an initial position, the needle-stopping member prevents backward moving the needle into the receptacle. The forward movement of the coupling appliance leads to the catheter emplacement and thereafter releases the stopping member thereby permitting the spring to retract the needle into the receptacle. As a result, the needle is removed from the catheter, wholly disposed within the receptacle and inaccessible for any contact with personnel. The needle removal from the catheter allows disconnecting the coupling appliance along with the receptacle and protected needle from the catheter. Subsequently, the catheter is occluded with a catheter cap. The disadvantage of the device is the restricted capability of the one-handed device control because the elongated stem is disposed within the receptacle and inaccessible for pushing by the fingers of user's hand holding the device. Another disadvantage is the considerable length of the receptacle and the sprig due to the necessity to hose the all needle inside of the receptacle to protect the needle sharp point. This leads to the inconvenience in the device holding by user's hand, heightens the material expenditure and total device cost, increases the package and shipping costs. Another disadvantage is the presence of a substantial friction between the elongated stem and the needle hub during the catheter placement caused with the spring action. As a result, the sensitivity of user's fingers needed for the estimation of the interaction between the catheter and patient's body is lowered. Besides, this friction worsens the device reliability creating the capability of the detail jamming. Another disadvantage is the relative complex device design caused with the disposition of the elongated stem within the receptacle, using the complicated design of the coupling appliance and carrying out the deepening within the receptacle for the engagement of the stopping member. Another disadvantage is the worsened observation of the flash chamber due to the spring disposition over the flash chamber. Another disadvantage is the impossibility of sufficient quick occlusion of the catheter opening after the catheter emplacement and the needle withdrawal leading to bleeding.

### SUMMARY OF THE INVENTION

The objective of the present invention is providing the capability of the device one-handed control during the catheter placement.

Another objective is reducing the device manufacturing cost due to decreasing the device holder and spring lengths and simplifying the device design.

Another objective is improving the convenience of the device use due to the capabilities of more convenient holding the device by user's hand, improving the user's sensation of the interaction between the device and patient's body, and improving the flash chamber observation.

Another objective is heightening the device reliability due to eliminating the substantial detail friction and jamming.

Another objective is providing the capability of the catheter occlusion immediately after the catheter emplacement and needle withdrawal to avoid the catheter bleeding. The noted objectives of the present invention are accomplished with the catheter placement device comprising a catheter and a catheter introducer. The catheter includes an axial through channel and a catheter hub with a female part of a luer-type connector. The catheter introducer has a needle unit and a telescopic protector. The needle unit includes a needle with a sharpened tip and a needle hub, which is located at a needle proximal end and includes a flash chamber. The telescopic protector includes a hollow elongated holder and a guard unit having a guard for protecting the needle sharpened tip after the catheter emplacement, as well as a telescoping guard carrier whereon the guard is located and which is slidably disposed in guides formed by the elements of the holder. Also there is a retracting spring, disposed inside of the holder between the elements of the holder and needle hub and designed to urge the needle hub to the holder proximal end. The needle hub is hosed in the holder and capable of displacing under the action of the retracting spring during the transposition of the needle unit from an extended position wherein the needle hub is disposed adjacent to a holder distal end to a retracted position wherein the needle hub is disposed adjacent to a holder proximal end. In the extended position, retaining means provides the releasable engagement of the needle unit with the holder at a holder distal end. The retracting spring is actuated by a triggering means, which is designed to disengage the needle unit from the distal holder end allowing the spring to transpose the needle unit to the retracted position. The device is provided with a locking means for locking the guard unit in a full length position wherein the guard unit extends from the holder in a distal direction as a result of the guard unit transposition relative to the holder from a short length position.

The catheter placement device has an initial and a protected positions.

In the initial position, the catheter placement device is ready for the catheter placement and the needle unit is in the extended position wherein the needle extends through the catheter channel so that the needle sharpened tip protrudes distally from a catheter distal end and the needle hub is disposed adjacently to the holder distal end. Besides, the retracting spring is held in a compressed state between the holder and the needle hub by the retaining means, the guard unit is in the short length position, and the guard is releasably coupled with the catheter hub and disposed adjacently to the holder distal end. Therewith, the retaining means provides the releasable engagement of the needle unit in the extended position with the holder at the holder distal end.

In the protected position, the guard unit is in the full length position in which the guard is displaced from the holder in a distal direction as a result of the catheter emplacement, and the guard unit is fixed to the holder by the locking means. Therewith, transposing the guard unit from the short length position to the full length position is accomplished manually during the process of the catheter placement. Besides, the retaining means is in a disengagement state, the retracting spring is in a minimal stressed state, and the needle unit is in the retracted position whereto the needle unit is transposed by the retracting spring during spring's transposition from the compressed state to the minimal stressed state, the needle sharpened tip is disposed inside of the guard, and the needle hub is rested against a stopper adjacently to the holder proximal end.

Thus, the disposition of the needle unit in the retracted position and the guard unit in the full length position provide protecting the needle sharpened tip by the guard. It means that protecting the needle sharpened tip is accomplished partly as the result of manual transposing the guard unit relative to the holder from the short length position to the full length position and partly as the result of automatic retracting the needle unit into the retracted position by the retracting spring. This allows using the holder of the relatively short length, which is less than the length of the needle unit. Correspondently, the length of the retracting spring also is shortened. Decreasing the length of the holder and retracting spring enables reducing the device manufacturing cost, as well as the package and shipping costs. Besides, this also provides more convenient holding the device by user's hand.

The retaining means includes at least one resilient retaining latch and at least one retaining ledge one of which is disposed on the holder and another on the needle hub so that in the initial position the retaining latch is in an engagement with the retaining ledge holding the needle unit in the needle extended position. The actuation of the spring is accomplished as a result of disengaging the retaining latch from the retaining ledge by the triggering means having at least one triggering member adapted to urge the retaining latch out of the engagement with the retaining ledge. Therewith, the triggering member interacts with the retaining latch and urges it out of the engagement with the retaining ledge at the last portion of the guard unit movement from the short length position to the full length position. Consequently, the retaining means does not offer the resistance to the guard unit during the most part of its movement. This eases the catheter introduction and improves the user's sensation of the interaction between the device and patient's body.

In version embodiment, the catheter placement device has the retaining means including a resilient retaining latch disposed on the needle hub and a retaining ledge located on the holder, therewith the retaining latch and the needle hub are made as an integral part. The guard carrier is made in the form of an elongated bar movably disposed in the holder guide, and the triggering member is made as a cam disposed on the elongated bar. The holder has an upper slit wherein the cam is moved and the retaining ledge is made as two shoulders located inside the holder upper slit on its lateral walls and defining a passageway for the cam, which passes between the shoulders forcing down the retaining latch and disengaging it from the retaining ledge. Thus, the triggering means is automatic and designed for the passive spring actuation as a result of disengaging the retaining latch from the retaining ledge automatically during the catheter placement before disconnecting the catheter introducer from the catheter. The passive spring actuation means that the transition of the catheter placement device from the initial position to the protected position does not demand any actions except the manipulation for the catheter emplacement.

In another version embodiment, the triggering means is an active spring actuating mechanism wherein the triggering member is made as a manually driven member in the form of a knob, which is deactivated in the initial position and after the transposition of the guard unit into the full length position the pressure onto the knob by operators finger disengages the retaining latch from the retaining ledge thereby releasing the retaining spring. Thus, there are two strongly separated operation stages: the transposition of the guard unit to the full length position is the first stage, and the needle transposition to the retracted position by spring is the second stage. The physician chooses the moment of the spring actuation, which is the second stage beginning.

The choice of the device having the passive or active version of the retracting spring actuation depends on the personal preference of the physician. In the both noted version embodiments, the accidental premature actuation of the retracting spring is eliminated due to that the triggering member is disposed beyond the zone of the retaining latch location almost up to the completion of the catheter placement. Moreover, the reuse of the device also is eliminated due to the presence of a securing means fixing the needle unit in the retracted position after the needle unit retraction by the retracting spring. The securing means includes a securing members disposed on the holder at its proximal end and on the needle unit. In version embodiment, the securing means includes a securing ledge disposed on the holder and adapted for engagement with the retaining latch located on the needle hub when the needle hub gets into the retracted position. In another version embodiment, the securing means includes a securing shield disposed at the holder proximal end and eliminating the accessibility to the needle hub in its retracted position, thereby preventing the needle unit from displacing to the extended position.

The catheter placement device includes an operating means for the catheter advance into a patient's vein and manual transposing the guard unit from the short length position into the full length position, and a connecting means providing a releasable coupling of the catheter hub and the guard up to the transposition of the guard unit into the retracted position. The connecting means includes the contacting friction surfaces of the catheter hub and the guard providing their coupling by the friction forces. In another embodiment, the connecting means is made as a thread connection of the catheter hub and the guard.

In version embodiment, the operating means is designed for the one-handed catheter emplacement and provides for the disposition of the guard carrier externally at the top of the holder and includes a plurality of small projections, which are located on an upper surface of the guard carrier and accessible for operators finger control from above. This substantially heightens the convenience of the device operation. Besides, the external disposition of the guard carrier simplifies the device design thereby reducing its cost and eliminates the substantial needle hub friction and jamming in the comparison with the prior art version. This substantially heightens the device reliability.

In the device version including the guard unit designed for the two-handed catheter emplacement, the catheter hub fulfils a role of the operating means.

The catheter placement device includes a catheter cap for occluding a proximal opening of the catheter hub after the device transition into the protected position and disconnecting the catheter introducer from the catheter. The catheter cap is detachably positioned on the catheter introducer so that, after the needle distal sharp tip protection, the catheter cap is accessible for occluding the catheter hub proximal opening without previous detachment of the catheter cap from the catheter introducer. The catheter cap has a conical obturator and is positioned at the holder proximal end so that the conical obturator is directed proximally and there is a mounting means allowing mounting/detaching the catheter cap on/from the holder. The presence of the catheter cap in the catheter placement device allows the catheter occlusion immediately after the catheter emplacement and needle withdrawal thereby avoiding the catheter bleeding.

The guard carrier in the short length position is located above the flash chamber and is provided with a window to improve an observation of the flash chamber. Moreover, the retracting spring in the initial position is disposed proximally of the distal portion of the flash chamber thereby also improving the flash chamber observation.

### BRIEF DESCRIPTION OF THE DRAWINGS

FIGS. 1 to 12 show the catheter placement device with a passive automatic actuation of the retracting spring by the cam disposed on the resilient arm of the guard carrier, wherein:

FIG. 1 shows the general view of the catheter placement device in the initial position.

FIG. 2 shows the longitudinal cross section of the catheter placement device in the initial position.

FIGS. 3, 4 show the transversal cross sections of the catheter placement device in the initial position.

FIG. 5 shows the back view of the catheter placement device.

FIG. 6 shows the longitudinal cross section of the catheter placement device after getting the guard unit into the full length position but before the retracting spring actuation.

FIG. 7 shows the longitudinal cross section of the catheter placement device at the moment of the retracting spring actuation.

FIG. 8 shows the longitudinal cross section of the catheter placement device after the retracting spring actuation and transposing the needle unit into the retracted position.

FIG. 9 shows the longitudinal cross section of the catheter placement device immediately after disconnecting the catheter from the catheter introducer.

FIG. 10 shows the longitudinal cross section of the catheter placement device after rotating the disconnected catheter introducer through 180° to begin the occlusion of the catheter proximal opening with the catheter cap.

FIG. 11 shows the longitudinal cross section of the catheter placement device at the moment of mounting the catheter cap onto the catheter with using the catheter introducer as a catheter cap holder.

FIG. 12 shows the longitudinal cross section of the catheter placement device after the emplacement of the catheter cap and removing the catheter introducer.

FIGS. 13 to 15 show the catheter placement device with the passive automatic actuation of the retracting spring by the resilient arm disposed on the guard carrier, wherein:

FIG. 13 shows the longitudinal cross section of the catheter placement device in the initial position.

FIG. 14 shows the longitudinal cross section of the catheter placement device at the beginning of the resilient arm operation.

FIG. 15 shows the longitudinal cross section of the catheter placement device at the moment of the retracting spring actuation with the resilient arm.

FIGS. 16 to 19 show the catheter placement device having an active spring actuating mechanism with the manual knob disposed on the guard carrier, wherein:

FIG. 16 shows the longitudinal cross section of the catheter placement device in the initial position.

FIG. 17 shows the longitudinal cross section of the catheter placement device wherein the guard unit is in the retracted position and the manual knob is disposed above the retaining latch and ready for the retracting spring actuation.

FIG. 18 shows the longitudinal cross section of the catheter placement device at the moment of the user's pressure onto the manual knob and disengaging by it the retaining latch from the retaining ledge.

FIG. 19 shows the longitudinal cross section of the catheter placement device immediately after the retracting spring actuation, the needle unit retraction into the retracted position and fixing there by the securing means.

### DETAILED DESCRIPTION OF THE INVENTION

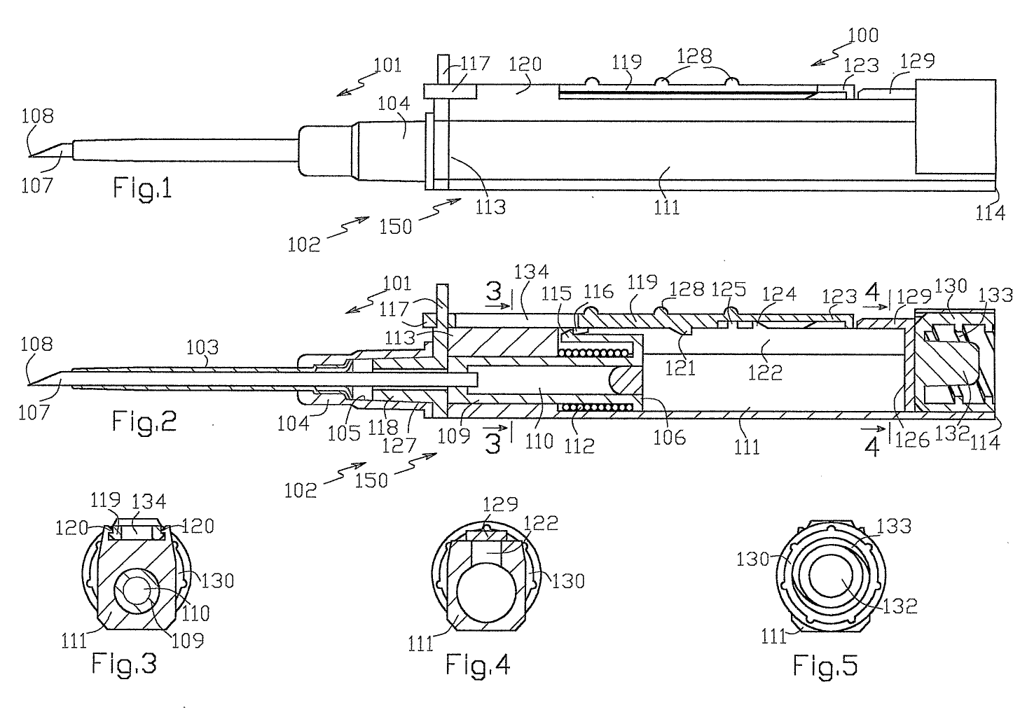

The detailed description of the present invention is offered with the references made to the enclosed drawings in FIGS. 1 to 19.

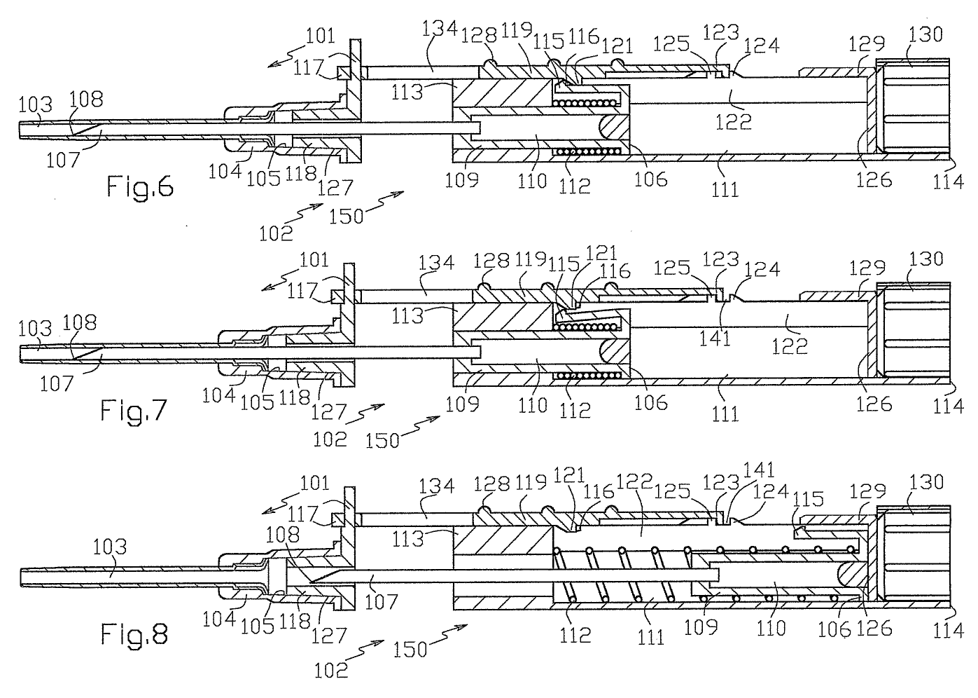

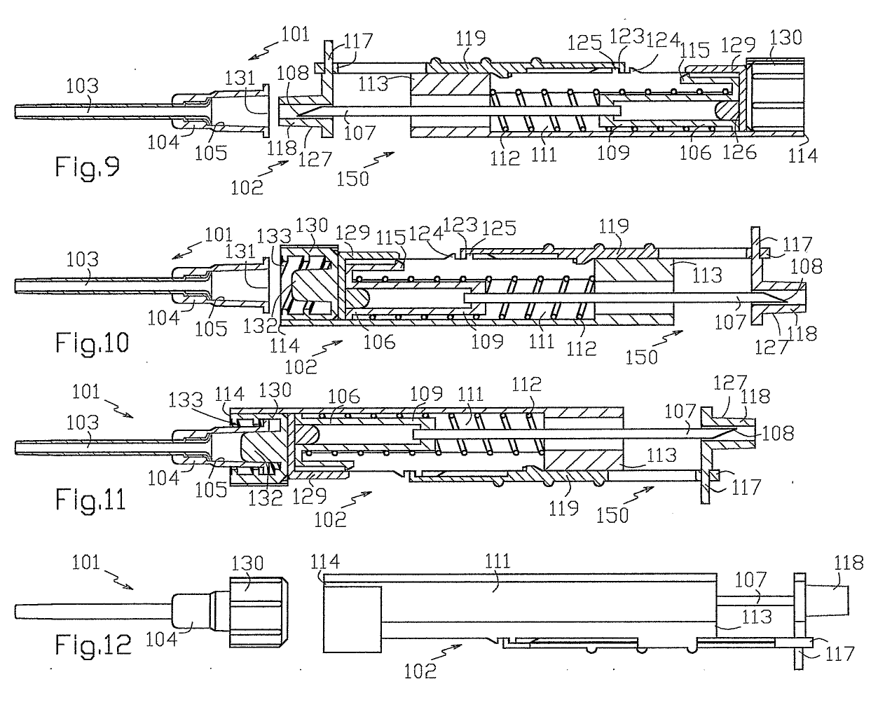

FIGS. 1 to 12 show catheter placement device 100 comprising catheter 101 and catheter introducer 102. Catheter 101 has axial through channel 103 and catheter hub 104 with female connector 105. Catheter introducer 102 has needle unit 106 and telescopic protector 135. Telescopic protector 150 has hollow elongated holder 111 and guard unit 117 including guard 118 and telescoping guard carrier 119 whereon guard 118 is located. Guard carrier 119 is made in the form of an elongated bar slidably disposed in guide 120 formed by the elements of holder 111. Needle unit 106 comprises needle 107 with sharpened tip 108 and needle hub 109, which is located at a needle proximal end and includes flash chamber 110. Needle hub 109 is movably hosed in holder 111. Retracting spring 112 disposed in holder 111 between the elements of needle hub 109 and holder 111 adapted to move needle unit 106 from an extended position (see FIGS. 2, 6, 7), wherein needle hub 109 is disposed adjacently to holder distal end 113, to a retracted position (see FIGS. 8, 9), wherein needle hub 109 is disposed adjacently to holder proximal end 114. Catheter introducer 102 includes a retaining means comprising resilient retaining latch 115 made as an integral part of needle hub 109 and retaining ledge 116 positioned on holder 111 (see FIG. 2). In version embodiment (not shown), the resilient retaining latch is disposed on the holder and the retaining ledge is disposed on the needle hub. In the device initial position (see FIG. 2), retaining members 115, 116 provide the releasable engagement of needle unit 106 with holder 111 at holder distal end 113, thereby holding needle unit 106 in the extended position and retracting spring 112 in a compressed state. Catheter introducer 102 is provided with a triggering means having a triggering member made as a cam 121 disposed on guard carrier 119. Therewith, holder 111 has upper slit 122 wherein cam 121 is moved, retaining ledge 116 is made as two shoulders (shown only one of them) located inside holder upper slit 122 on its lateral walls and defining a passage-way for cam 121, which passes between the shoulders forcing down retaining latch 115 (see FIG. 7) and disengaging it from retaining ledge 116. The triggering means is designed to disengage needle unit 106 from distal holder end 113 to actuate retracting spring 112. After the actuation, retracting spring 112 transposes needle unit 106 to the retracted position (see FIGS. 8, 9). Triggering member 121 is adapted to disengaging retaining latch 115 from retaining ledge 116 automatically during the catheter placement before disconnecting catheter introducer 102 from catheter 101. Catheter introducer 102 is provided with a locking means for locking guard unit 117 in a full length position (see FIGS. 6 to 9) wherein guard unit 117 extends from holder 111 in a distal direction as a result of the guard unit transposition distally relative to holder 111 from a guard unit short length position. The locking means comprises resilient locking arm 123 located on guard carrier 119, and locking ledge 124 positioned on holder 111. When guard unit 119 moves to the full length position, locking arm 123 is biased into a locking engagement with locking ledge 124 and this locking engagement prevents guard unit 119 from displacing proximally relative to holder 111. Beginning from this locking engagement, guard unit 119 is in the full length position. Therewith, the retaining means engages the needle unit with the holder in the needle extended position at least up to locking the guard unit in the full length position. Besides, there is guard unit limiter 125 restricting the displacement of guard unit 117 distally relative to holder 111 after the mentioned locking engagement.

The elements of catheter placement device 100 have an initial and a protected positions.

In the initial position (see FIGS. 1, 2), catheter introducer 102 is ready for the catheter placement and needle unit 106 is in the extended position wherein needle 107 extends through catheter channel 103 so that needle sharpened tip 108 protrudes distally from the catheter distal end and needle hub 109 is disposed adjacently to holder distal end 113. Besides, retracting spring 112 is held in a compressed state between holder 111 and needle hub 109 by retaining members 115, 116, guard unit 117 is in the short length position, and guard 118 is releasably coupled with catheter hub 104 and disposed adjacently to holder distal end 113. Therewith, the retaining means provides the releasable engagement of needle unit 106 in the extended position with holder 111 at holder distal end 113.

In the protected position (see FIG. 8), guard unit 117 is in the full length position wherein guard 118 is displaced from holder 111 in a distal direction as a result of the catheter emplacement, and guard unit 117 is locked relative to holder 111 by locking members 123, 124. Therewith, transposing guard unit 117 from the short length position to the full length position is accomplished manually during the process of the catheter placement. Besides, the retaining latch 115 and retaining ledge 116 are in a disengagement state, retracting spring 112 is in a minimal stressed state, and needle unit 106 is in the retracted position whereto needle unit 106 is transposed by retracting spring 112 during its transition from the compressed state to the minimal stressed state, needle sharpened tip 108 is disposed inside of guard 118, and needle hub 109 is rested against stopper 126 adjacently to holder proximal end 114.

Thus, the disposition of needle unit 106 in the retracted position and guard unit 117 in the full length position provide protecting needle sharpened tip 108 by guard 118 (see FIGS. 8 to 12). It means that the protection of needle sharpened tip 108 is accomplished partly at the expense of manual transposing guard unit 117 relative to holder 111 from the short length position to the full length position and partly at the expense of automatic retracting needle unit 106 into the retracted position by retracting spring 112. This allows using holder 111 of the relatively short length, which is less than the length of needle unit 106. Correspondently, the length of retracting spring 112 also is shortened. Decreasing the length of the holder and retracting spring enables reducing the device manufacturing cost, as well as the package and shipping costs. Besides, this also provides more convenient holding the device by user's hand.

The sequence of the retracting spring actuation and getting the guard unit into the full length position is determined by a triggering index equal to the difference between the distance passed by the guard unit from the short length position to the moment of the retracting spring actuation and the distance passed by the guard unit from the short length position to its full length position. As applied to catheter introducer 102, the triggering index is positive that provides getting guard unit 117 into the full length position before actuating retracting spring since, after locking guard unit 117, it must additionally pass gap 141 to actuate retracting spring 112.

Catheter introducer 102 includes an operating means for the advance of catheter 101 into a patient's vein and the manual transposition of guard unit 117 from the short length position into the full length position. The operating means provides for the disposition of guard carrier 119 externally at the top of catheter introducer 102 and includes a plurality of small projections 128, which are located on an upper surface of guard carrier 119 and accessible for operator's finger control from above. As a result, the operating means allows the one-handed catheter emplacement. This improves the convenience of the device operation. Besides, the external disposition of guard carrier 119 simplifies the device design thereby reducing its cost and eliminates the substantial needle hub friction and jamming in the comparison with the prior art version. This substantially heightens the device reliability.

The presence of the operating means for the one-handed catheter emplacement does not eliminate the two-handed catheter emplacement with using the catheter hub as the operating means.

Catheter placement device 100 has a connecting means providing a releasable coupling of catheter hub 104 and guard 118 up to the transposition of needle unit 106 into the protected position. The connecting means includes contacting friction surfaces 105, 127 of catheter hub 104 and guard 118, respectively, providing their coupling by the friction forces.

The accidental premature actuation of retracting spring 112 is eliminated due to that triggering member 121 is disposed beyond the zone of the location of retaining latch 115 during the all user's manipulations, except the position shown in FIG. 7 wherein retracting spring 112 should be actuated and triggering member 121 is disposed above retaining latch 115. Moreover, the reuse of catheter introducer 102 also is eliminated due to the presence of a securing means preventing the displacement of needle unit 106 distally from the retracted position after retracting needle unit 106 by retracting spring 112. The securing means includes securing shield 129 disposed at holder proximal end 114 and eliminating the accessibility to needle hub 109 in its retracted position, thereby preventing needle assembly 106 from being displaced to the extended position.

Catheter introducer 102 includes catheter cap 130 for occluding proximal opening 131 of catheter hub 104 after disconnecting catheter introducer 102 from catheter 101 (see FIG. 9). Catheter cap 130 is detachably mounted on catheter introducer 102 so that, after the protection of needle distal sharp tip 108, catheter cap 130 is accessible for occluding the catheter hub proximal opening 131 without previous detachment of the catheter cap from the catheter introducer. Catheter cap 130 has conical obturator 132 and inner thread 133, is positioned at holder proximal end 114 so that conical obturator 132 initially is directed proximally (see FIG. 2), and there is a mounting means allowing mounting/detaching catheter cap 130 on/from holder 111. As applied to catheter introducer 102, catheter cap is mounted on holder 111 by means of the friction forces. After disconnecting catheter introducer 102 from catheter 101, user rotates catheter introducer 102 through 180° (see FIG. 10) and screws catheter cap 130 on catheter hub 104 using catheter introducer 102 as a catheter cap holder (see FIG. 11). Then, user disconnects catheter introducer 102 from catheter cap 130. The presence of the catheter cap in the catheter placement device allows the catheter occlusion immediately after the catheter emplacement and needle withdrawal thereby avoiding the catheter bleeding.

Guard carrier 119 in the short length position (see FIGS. 1, 2) is located above flash chamber 110 and is provided with window 134 to improve the observation of flash chamber 110. Moreover, retracting spring 112 in the initial position is disposed proximally of the distal portion of flash chamber 110 thereby also improving the flash chamber observation.

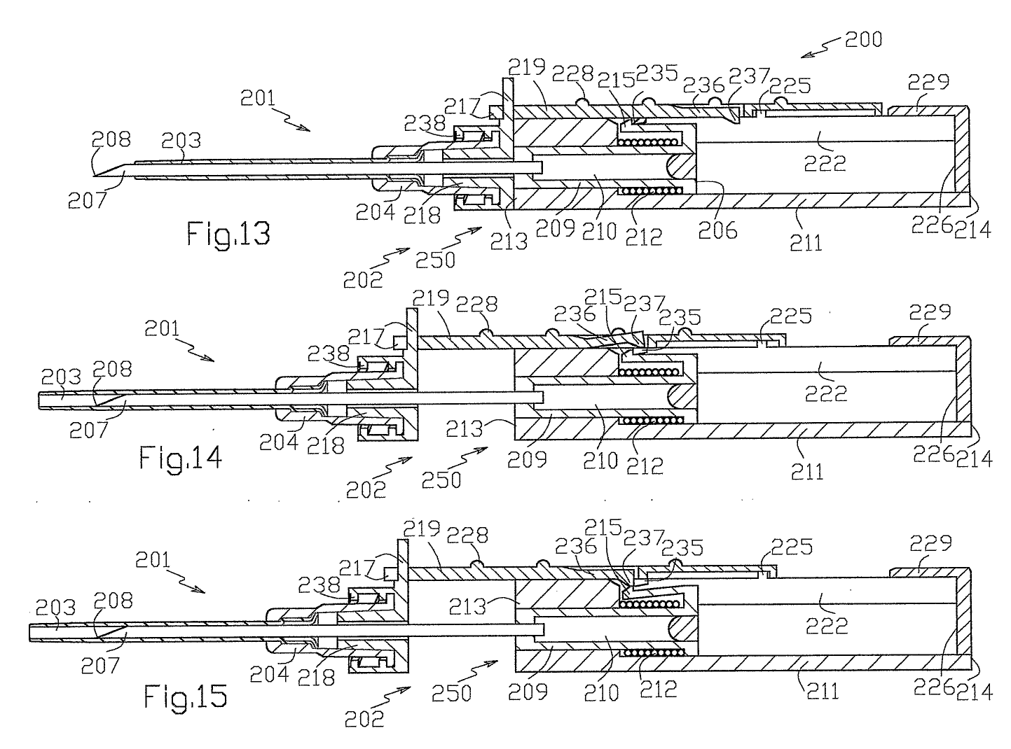

FIGS. 13 to 15 show the version embodiment of catheter placement device 200 with a passive actuation of retracting spring 212. Device 200 has many details identical to one of above device 100 and having the same design, operation, advantages and two last designation numerals. At the same time, device 200 has the distinctive features. Specifically, the retaining ledge and the locking ledge are made as an integral retaining-locking ledge in the form of cross-beam 235 located on holder 211 transversely relative to holder upper slit 222 and engaged with retaining latch 215 in the initial position (see FIG. 13). The triggering member is made as resilient arm 236 fulfilling also a role of the locking latch, disposed on elongated bar 219 and terminating in cam 237. The latter is movable in holder upper slit 222 and adapted to cross over cross-beam 235 and jumping off cross-beam 235 to force down retaining latch 215 during transposing guard unit 217 from the short length position to the full length position (see FIG. 14, 15), thereby disengaging retaining latch 215 from cross-beam 235 and simultaneously engaging resilient arm 236 with cross-beam 235 to lock elongated bar 219 relative to holder 211. Therewith, guard unit limiter 225 restricts the displacement of guard unit 217 distally relative to holder 211 after mentioned locking the elongated bar 219. The triggering index of catheter introducer 202 is equal to zero that provides getting guard unit 217 into the full length position simultaneously with actuating retracting spring 212 since the moments of locking guard unit 217 and actuating the retracting spring 212 coincide. The connecting means of catheter placement device 200 providing the releasable coupling catheter hub 204 and guard 218 also relates to the distinctive features since it is made as thread connection 238.

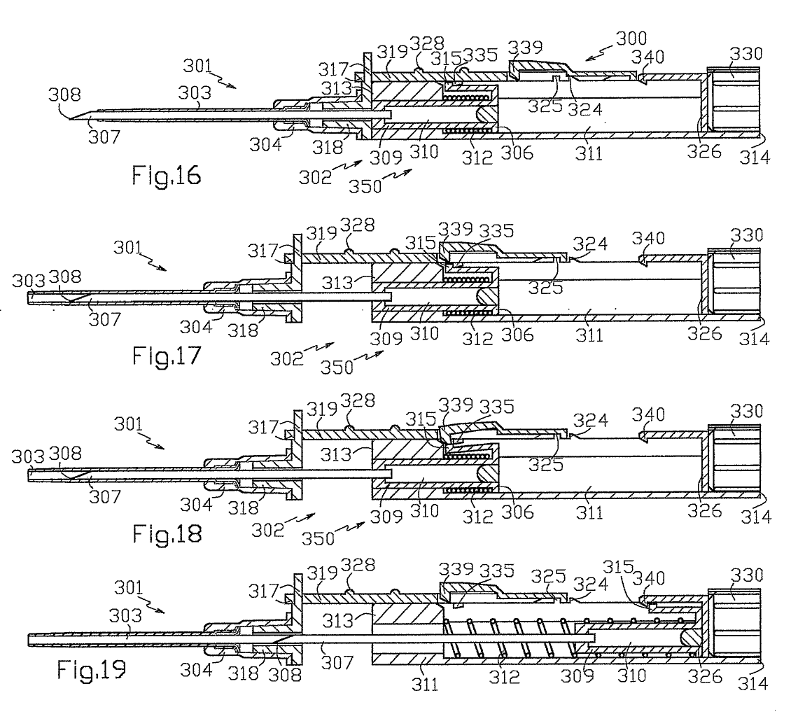

FIGS. 16 to 19 show the version embodiment of catheter placement device 300 with an active actuation of retracting spring 312. Device 300 has many details identical to one of above device 100 and having the same design, operation, advantages and two last designation numerals. At the same time, device 300 has the distinctive features. Specifically, its triggering means is made as an active spring actuating mechanism wherein the triggering member is made as a manually driven member in the form of knob 339, which is located on guard carrier 319 and deactivated in the initial position (see FIG. 16). After the transposition of guard unit 317 into the full length position (see FIG. 17), the pressure onto knob 339 by operator's finger (see FIG. 18) disengages retaining latch 315 from retaining ledge 335 thereby releasing retaining spring 312 (see FIG. 19). The choice of the devices with the automatic or manual version of the retracting spring actuation depends on the personal preference of the surgeon. The accidental premature actuation of retracting spring 312 is eliminated due to that knob 339 is disposed beyond the zone of the location of retaining latch 315 during the all user's manipulations, except the guard unit position wherein retracting spring 312 should be actuated and knob 339 is disposed above retaining latch 315. Moreover, the reuse of catheter introducer 302 also is eliminated due to the presence of the securing means fixing needle assembly 306 in the retracted position after the needle unit retraction by retracting spring 312. The securing means includes securing ledge 340 disposed on holder 311 at holder proximal end 314 and adapted for engagement with retaining latch 315 located on needle hub 309 when needle hub 309 gets into the retracted position. The triggering index of catheter introducer 302 is equal to zero since the guard unit position wherein guard unit 317 is locked and the guard unit position wherein knob 339 is located above retaining latch 315 allowing actuating the retracting spring 312 coincide.

In version embodiment (not shown), the manual knob and the retaining latch are made as an integral part, therewith the guard carrier blocks the accessibility to the knob up to the transfer of the guard unit into the full length position.

1. A catheter placement device, having:
a catheter having:
an axial through channel,
a distal end,
a proximal end,
a catheter hub attached at said catheter proximal end;
catheter introducer, including:
a needle unit, having:
a needle, having:
a distal end with a sharpened tip,
a proximal end,
a needle hub including a flash chamber and attached to said needle proximal end,
an extended position and
a retracted position;
a telescopic protector, including:
a guard unit, having:
a guard and
a guard carrier whereon said guard is placed,
a short length position and
a full length position;
a hollow elongated holder, having:
a distal end and
a proximal end;
said guard carrier placed outside of said needle hub and being in sliding engagement with a guide formed by elements of said holder;
a locking means for fixing said guard unit to said holder in said full length position;
said needle hub housed in said holder so that in said needle extended position said needle hub is disposed at said holder distal end, in said retracted position said needle hub is disposed at said holder proximal end, and said needle tip protrudes distally from said holder;
an operating means for manual displacement of said guard unit from said short length position to said full length position;
a retaining means for releasable engagement of said needle unit with said holder in said needle extended position so that said engagement continues at least up to locking said guard unit in said full length position;
a retracting spring for automatic displacement of said needle unit from said needle extended position to said needle retracted position;
an initial position wherein the device is ready for the catheter placement,
said needle unit is in said extended position, said needle sharpened tip protrudes distally from said catheter distal end, said guard unit is in said short length position,
said retaining means is in an engagement state holding said retracting spring in a compressed state between said holder and needle hub,
said guard is releasably coupled with said catheter hub and disposed adjacently to said holder distal end;
a protected position wherein
said guard unit is in said full length position as a result of the catheter emplacement and is fixed to said holder by said locking means;
said retaining means is in a disengagement state and said retracting spring is in a minimal stressed state;
said needle unit is in said retracted position and said needle sharpened tip is disposed inside of said guard;
an operating means for manual displacement of said guard unit from said short length position to said full length position;
a triggering means for disengagement of said needle unit from said holder distal end.

2. The catheter placement device of claim 1, wherein
said retaining means including at least one resilient retaining latch and at least one retaining ledge one of which is disposed on said holder and another on said needle hub so that in said initial position said retaining latch is in a retaining engagement with said retaining ledge holding said needle unit in said needle extended position, and an actuation of said retaining spring is caused by disengaging said retaining latch from said retaining ledge;
said triggering means having at least one triggering member adapted to urge said retaining latch out of said engagement with said retaining ledge;
said locking means including at least one resilient locking arm and at least one locking ledge one of which is disposed on said holder and another on said guard carrier so that when said guard unit is moved in a distal direction said locking arm is biased into a locking engagement with said locking ledge;
a connecting means providing a releasable coupling said catheter hub and said guard up to releasing said retracting spring.

3. The catheter placement device of claim 2, wherein
said guard carrier is made in the form of an elongated bar;
said triggering member is disposed on said guard carrier;
said retaining ledge is located on said holder;
said retaining latch is located on said needle hub.

4. The catheter placement device of claim 3, wherein
said triggering means is designed for a passive spring actuation;
said triggering member presents an automatic member disengaging said retaining latch from said retaining ledge automatically during emplacement of said catheter before disconnection of said catheter introducer from said catheter;
said triggering member is made as a cam disposed on said elongated bar;
said retaining latch and said needle hub are made as an integral part;
said holder has an upper slit wherein said cam is moved;
said retaining ledge is made in the form of two shoulders allowing passing said cam between said shoulders to force down said retaining latch and disengage it from said shoulders.

5. The catheter placement device of claim 3, wherein
said triggering means is designed for a passive spring actuation;
said triggering member presents an automatic member disengaging said retaining latch from said retaining ledge automatically during emplacement of said catheter before disconnection of said catheter introducer from said catheter;
said retaining latch and said needle hub are made as an integral part;
said holder has an upper slit;
said retaining ledge and said locking ledge are made as an integral retaining-locking ledge in the form of a cross-beam located on said holder transversely to said holder upper slit and engaged with said retaining latch in said initial position;
said triggering member fulfilling also a role of said locking latch is made as a resilient arm having a cam, adapted to cross over said cross-beam and jump off said cross-beam to force down said retaining latch during transposition of said guard unit from said short length position to said full length position, thereby disengaging said retaining latch from said cross-beam and simultaneously engaging said resilient arm with said cross-beam and fixing said guard unit to said holder.

6. The catheter placement device of claim 3, wherein:
said triggering means is designed for an active spring actuation;
said triggering member is a manually driven member having a knob, adapted for operators finger control;
said knob and said locking latch are made as an integral part;
said retaining ledge and said locking ledge are made as an integral retaining—locking ledge in the form of a cross-beam located on said holder transversely to said holder and engaged with said retaining latch in said initial position;
said triggering member fulfilling also a role of said locking latch is made as a resilient arm having a cam controlled by said knob and adapted to cross over said cross-beam, to lock said guard unit in said full length position and after this to force down said retaining latch and thereby to disengage said retaining latch from said cross-beam.

7. The catheter placement device of claim 2, wherein said retaining latch is disposed on said holder and said retaining ledge is disposed on said needle hub.

8. The catheter placement device of claim 2, wherein said operating means is designed for the one-handed catheter emplacement and includes said guard carrier disposed externally at the top of said holder and accessible for operator's finger control from above.

9. The catheter placement device of claim 2, wherein said connecting means includes contacting friction surfaces of said catheter hub and said guard providing their coupling by the friction forces.

10. The catheter placement device of claim 2, wherein said connecting means is made as a thread connection between said catheter hub and said guard.

11. The catheter placement device of claim 1, wherein there is a catheter cap for occluding said catheter hub after disconnecting said catheter introducer from said catheter, therewith said catheter cap is detachably positioned on said catheter introducer so that, after enclosing said needle sharpened tip by said guard, said catheter cap is accessible for occluding a proximal opening of said catheter hub without previous detachment of said catheter cap from said catheter introducer.

12. The catheter placement device of claim 11, wherein said catheter cap has a conical obturator and is positioned at said holder proximal end so that said conical obturator is directed proximally and there is a mounting means for mounting/detaching said catheter cap on/from said catheter introducer.

13. The catheter placement device of claim 2, wherein said guard carrier has a window to improve an observation of said flash chamber and in said short length position of guard unit said window is located above said flash chamber.

14. The catheter placement device of claim 2, wherein said flash chamber has a distal portion for blood visualization and said retracting spring in said device initial position is disposed proximally of said flash chamber distal portion thereby improving the blood indication.

15. The catheter placement device of claim 2, having a securing means preventing the displacement of said needle unit from said needle retracted position and said securing means includes securing members, one of which is disposed on said holder and another on said needle hub, and said securing members are adapted to the engagement of said needle unit with said holder in said retracted position.

16. A catheter placement device, including:
a catheter introducer,
a catheter with a catheter hub attached to said catheter introducer,
a catheter cap for occluding said catheter hub after disconnecting said catheter introducer from said catheter, therewith said catheter cap is detachably positioned on said catheter introducer so that, said catheter cap is accessible for occluding said catheter hub without previous detachment of said catheter cap from said catheter introducer.

17. The catheter placement device of claim 16, wherein
said catheter introducer has a proximal end,
said catheter cap has a conical obturator and is positioned at said catheter introducer proximal end so that said conical obturator is directed proximally and there is a mounting means for mounting/detaching said catheter cap on/from said catheter introducer.

18. A catheter placement device, including:
a catheter;
a catheter introducer, including:
a needle unit, having:
an extended position and
a retracted position;
a telescopic protector, having:
a guard unit, having:
a short length position and
a full length position;
a holder, having:
a distal end and
a proximal end;
said guard unit being in sliding engagement with a guide formed by elements of said holder;
an operating means for manual displacement of said guard unit from said short length position to said full length position;
a retracting spring for automatic displacement of said needle unit from said needle extended position to said needle retracted position;
a retaining means for releasable engagement of said needle unit with said holder distal end, including at least one resilient retaining latch and at least one retaining ledge one of which is disposed on said holder and another on said needle unit so that in said extended position of needle unit said retaining latch is in a retaining engagement with said retaining ledge, and an actuation of said spring is caused by disengaging said retaining latch from said retaining ledge;
a triggering means for disengagement of said needle unit from said holder distal end, having at least one triggering member adapted to urge said retaining latch out of said engagement with said retaining ledge.

19. The catheter placement device of claim 18, having:
a locking means for fixing said guard unit to said holder in said full length position;
said locking means including at least one resilient locking arm and at least one locking ledge one of which is disposed on said holder and another on said guard unit so that when said guard unit is moved in a distal direction said locking arm is biased into a locking engagement with said locking ledge; and
said triggering means and said locking means adapted to release said retracting spring after locking said guard unit in said full length position.
